(19) United States
(12) Patent Application Publication
(10) Pub. No.: US 2022/0134070 A1
(43) Pub. Date: May 5, 2022

(54) Micro-dosing Oil Dispenser

(71) Applicants: Kevinn Caytui Hirsch, London (GB); David Edward Conway, London (GB)

(72) Inventors: Kevinn Caytui Hirsch, London (GB); David Edward Conway, London (GB)

(21) Appl. No.: 17/085,355

(22) Filed: Oct. 30, 2020

(51) Int. Cl. A61M 35/00; B05C 17/005; B05C 11/10; B05C 17/01

(52) U.S. Cl. CPC A61M 35/003; B05C 17/00576; B05C 17/00516; A61M 2205/587; B05C 17/01; A61M 2202/04; A61M 2205/18; B05C 11/1034

(57) ABSTRACT

A micro-dosing oil dispenser with the ability to dispense multiple doses after the user rapidly depresses a dispensing button. The oil dispenser may vibrate upon completion of the doses. The oil dispenser may have a felt tip for better absorption.

### TECHNICAL FIELD

The present application relates to precise oil dispensing devices. More specifically, the present application relates to electronic transdermal oil extract dispensing devices.

### BACKGROUND

Oil dispensing medicament devices are common in the industry. Many devices have clumsy user experiences. Some of the causes of this clumsiness are poor control over multiple micro-doses and application of the medicaments. The present invention resolves these issues.

### SUMMARY OF THE INVENTION

The present application discloses a device that dispenses oil extracts for transdermal application. The device may allow a user to select several micro-doses at one application, and may vibrate upon completion of the application. The device may also have a felt tip for contact with the user's skin to enhance absorption.

### BRIEF DESCRIPTION OF DRAWINGS

FIG. 1 is prospective view of the exterior of the micro-dosing oil dispenser without the cap installed.

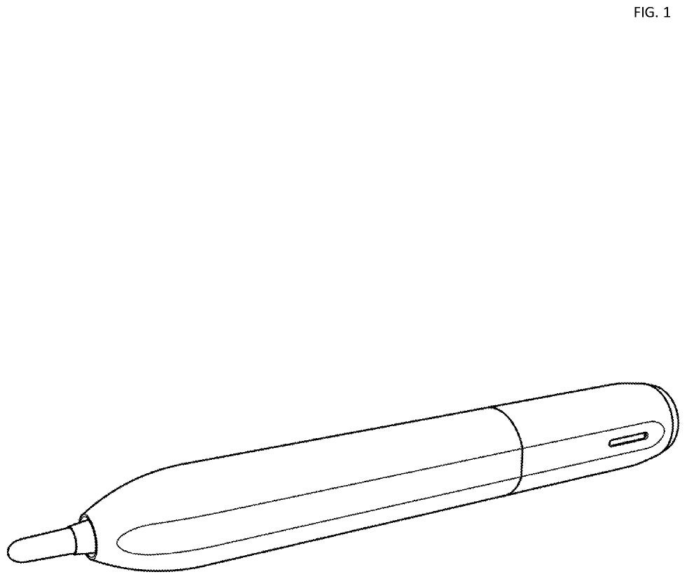

FIG. 2 is a prospective view of the exterior of the micro-dosing oil dispenser with the cap installed.

FIG. 3 is a side view of the interior parts of the micro-dosing oil dispenser.

### DETAILED DESCRIPTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limits include both numbers. For example, “25 to 100” means a range whose lower limit is 25 and upper limit is 100, and includes both 25 and 100.

The present application discloses a micro-dosing oil dispenser device. The device may be electronic which may enable it to provide advantages to the user experience. Some of these advantages are described below.

The device may have a memory chip which allows it to be pre-programmed to dispense certain quantity of oil. Different versions of the device may be available that have larger or smaller quantities per dose. In any version of the device, the user may select any number of doses per application. The user may not need to wait until each dose is dispensed before continuing to select more doses. Rather, the user may select as many doses as needed all at once by pressing the appropriate button as many times as the dose is desired. The user may then place the device on its skin and allow the oil to dispense. When the device has finished dispensing all doses, the device may vibrate to signal to the user that the application is finished.

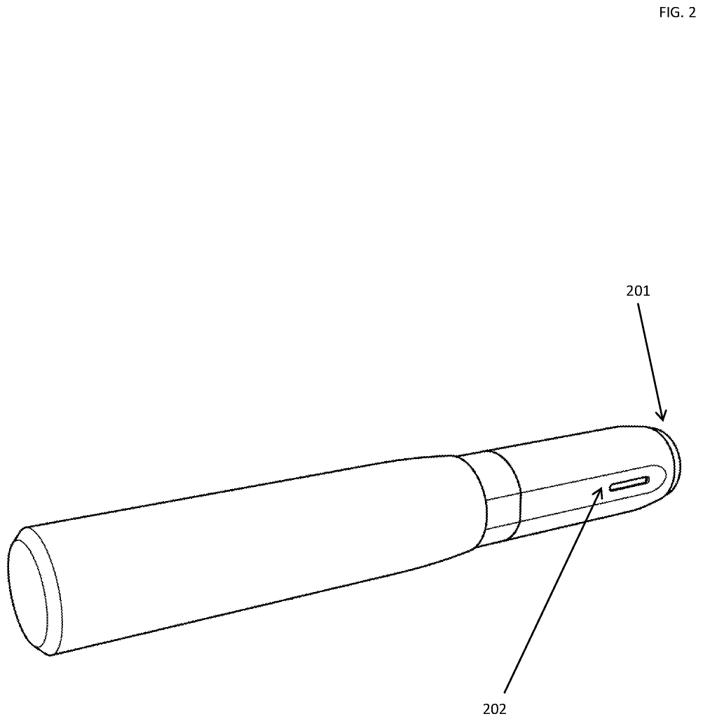

The device may be activated by engaging a button at the distal side of the device, shown in FIG. 2 as 201. This button may trigger programmed responses from the device's printed circuit board (“PCB”). The PCB may then activate an LED light, 202, or display which may visually indicate to the user that the unit is functioning. For example, once the button is engaged, the LED may turn amber while it waits for the user to complete its dose selection. The LED may then turn green while dispensing oil. Finally the LED turns off and the device vibrates when the application is complete. Alternatively, if the dispenser is empty, the LED may turn red for 15 seconds and then turn off. If, at the end of the application, the device is low on oil, the LED may flash red then switch off.

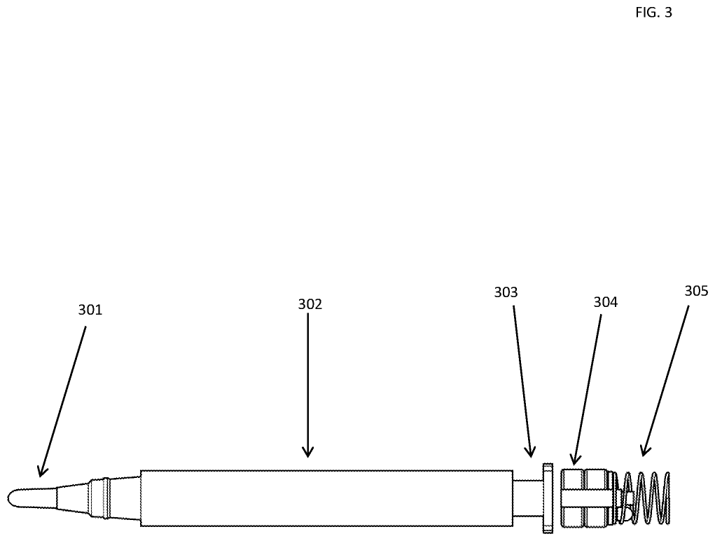

The button, 201, mentioned above, may have multiple purposes. In addition to activating an electronic response from the PCB, the button may work dispense the device's medicaments. The button may be connected to a plunger, shown as 303 on FIG. 3, which ejects the medicaments from the cartridge, 302. The plunger is ratcheted down as more and more as the button is pushed and product is dispensed. The button may be able to return to its original position by the ratcheting system and springs, shown as 305.

The device may also have batteries, 304, in order to operate. The device may also have a motor to allow it to vibrate.

The PCB may have memory elements. These elements may be able to track the number of doses the device has performed in order to determine when the oil containing cartridge may be low, half-full, and empty.

The device may have replaceable oil cartridges. Each cartridge may contain two hundred and fifty doses. Different oil cartridges may be available with different formulations. For example, one formulation may be full-spectrum CBD oil. Another formulation may be pure CBD oil. Another formation may be transdermal vitamins.

At the proximal side of the cartridge, there may be a felt tip, 301, for contact with the user's skin. This felt tip may be permanently fixed to the cartridge and may be discarded when the cartridge is empty. A cap may cover the felt tip when the device in not in use.

The invention claimed is:

1. A medicament dispensing device comprising:
a body housing a cartridge;
wherein medicament is ejected from the cartridge using a plunger depressed at the far end of the body; and
wherein the plunger is attached to a shaft with a ratcheting mechanism.

2. The device of claim 1, further comprising:
a button which is depressed at the end of the shaft of the plunger which activates a circuit board capable of storing the number of presses to the button in its memory;
wherein the circuit board may activate a motor in order to cause the device to vibrate;
wherein the circuit board may activate an LED.

3. The device of claim 1, further comprising a felt tip through which medicament is dispensed.
